(19) United States
(12) Patent Application Publication
(10) Pub. No.: US 2017/0100270 A1
(43) Pub. Date: Apr. 13, 2017

(54) SLEEVES FOR POSITIONING A STENT ON A DELIVERY BALLOON CATHETER SYSTEM

(71) Applicant: OrbusNeich Medical, Inc., Ft. Lauderdale, FL (US)

(72) Inventors: Robert J. Cottone, Davie, FL (US); Shusheng Ye, Davie, FL (US)

(21) Appl. No.: 15/279,030

(22) Filed: Dec. 12, 2016

Related U.S. Application Data
(63) Continuation of application No. 12/464,042, filed on May 11, 2009, now abandoned.
(60) Provisional application No. 61/052,187, filed on May 10, 2008.
(60) Provisional application No. 61/052,171, filed on May 10, 2008.

(51) Int. Cl. A61F 2/958

(52) U.S. Cl. CPC A61F 2/958; A61F 2002/9583

(57) ABSTRACT

The present disclosure provides one or more sleeves for positioning a stent on a stent delivery system, such as a balloon catheter. The sleeve wraps around the catheter. One or two sleeves are positioned adjacent to one or both ends of the stent without overlapping with the stent. The profile of the stent delivery system is not increased by the sleeves.

### CROSS REFERENCE TO RELATED APPLICATION

This application is a continuation of U.S. patent application Ser. No. 12/464,042 filed on May 11, 2009; which claims benefit of Provisional Application Nos. 61/052,171 and 61/052,187 filed on May 10, 2008.

### FIELD OF THE INVENTION

The present invention relates to the field of inflatable, percutaneous transluminal coronary angioplasty (PTCA) balloon catheters used to deliver and deploy prosthetic vascular devices or stents at specific sites within the vascular system of a patient.

### BACKGROUND OF THE INVENTION

Atherosclerosis is one of the leading causes of death and disability in the world. Atherosclerosis involves the deposition of fatty plaques on the luminal surface of arteries, which in turn causes stenosis, i.e., narrowing of the artery. Ultimately, this deposition blocks blood flow distal to the lesion causing ischemic damage.

Angioplasty has gained wide acceptance for treating various types of vascular disease. In particular, angioplasty is considered effective for opening stenotic areas in the coronary arteries, as well as in other areas of the body (Boden et al., Optimal Medical Therapy with or without PCI for Stable Coronary Disease, N. Engl. J. Med. 2007, 356:1503-1516). Percutaneous transluminal coronary angioplasty (PTCA) involves the use of a dilatation catheter which carries an inflatable balloon at the distal end of the catheter. A hollow guide catheter is initially placed in the femoral artery of the patient through a percutaneous cut-down. The guide catheter is then advanced along the descending aorta over the aortic arch and into the ascending aorta that leads from the heart. Under fluoroscopy, the physician then used the guide catheter to guide a dilatation catheter through the vascular system until the balloon is positioned at the site of stenosis (U.S. Pat. No. 5,976,120). The balloon is then inflated by fluid supplied under pressure through the inflation lumen, which extends from a proximal end of the catheter to a distal end of the catheter, and finally to the internal lumen of the balloon. The inflation of the balloon causes stretching of the artery, pressing of the lesion onto the artery wall, thus reestablishing an acceptable blood flow through the artery. An expandable stent may be delivered through the balloon catheter. After the balloon catheter is retracted from the body, the stent is deployed at the site of stenosis to maintain patency of the artery.

When the stent is delivered to a target site, the stent should remain tightly positioned on the stent delivery system until the site of stenosis is reached. A serious problem associated with many existing stent delivery systems is that the stent dislodges when the stent delivery system traverses through the tortuous vascular system (U.S. Pat. No. 6,589,274). Moreover, the stent may dislodge when it tracks over a guide wire or traverses the guiding catheter. Dislodgement of the stent during delivery is accompanied by significant morbidity and mortality. Cantor et al. Failed coronary stent deployment. American Heart Journal. 136(6):1088-1095 (1998).

Various stent retention devices have been developed to position the stent in place on the stent delivery system. For example, U.S. Pat. Nos. 6,589,274 and 6,221,097 disclose a pair of elastic retaining sleeves (caps) positioned near the proximal and distal ends of the stent. When the retaining sleeves and the stent expand together with the balloon, the ends of the stent slide out from under the retaining sleeves. The stent is then deployed. In many of these stent delivery devices, the outer diameter of the retaining sleeves is greater than the outer diameter of the stent. As a result, the profile of the stent delivery system is increased. In addition, after the balloon is fully expanded at the target site, the expanded retaining sleeves may also come into contact with the vessel wall, thus causing damage to the vessel. Another problem associated with such devices is that the stent may fail to completely exit from underneath the retaining sleeve during deployment.

Therefore, there is a need to develop a simple and effective stent retention system to prevent the stent from dislodging during delivery to the target site.

### SUMMARY OF THE INVENTION

It is an object of the present invention to provide one or more sleeves for maintaining the position of a stent on a stent delivery system when the stent is delivered to a target site. The stent delivery system comprises a catheter which has a region for mounting a stent and at least one sleeve made from expandable material. The sleeve is mounted on the catheter and is positioned adjacent to the stent. In one embodiment, the sleeve is in direct contact with the stent. In another embodiment, the stent is positioned between two sleeves. The outer diameter of the end of the unexpanded sleeve adjacent to the stent may be equal to or less than the outer diameter of the unexpanded stent mounted on the catheter.

The thickness of the sleeve wall at the end adjacent to the stent may range from about 0.03 mm to about 0.25 mm. The sleeve wall may decrease in thickness from the end of the sleeve adjacent to the stent to the end of the sleeve distal to the stent. The thickness of the sleeve wall may also be constant across the length of the sleeve. The edge of the sleeve adjacent to the stent may be flanged, flared, beveled, rounded or straight. The inner diameter of the sleeve may be constant across the entire length of the sleeve when the sleeve is in an unexpanded state. The inner diameter of the sleeve may decrease from the end of the sleeve adjacent to the stent to the end of the sleeve distal to the stent when the sleeve is in an unexpanded state. In one embodiment, the decrease is linear. In another embodiment, the inner diameter of the sleeve is constant across a first section of the sleeve beginning from the end of the sleeve adjacent to the stent, then decreasing across a second section of the sleeve adjacent to the first section and then remains constant across a third section adjacent to the second section. The third section is closest to the end of the sleeve distal to the stent. The inner diameter of the third section is less than the inner diameter of the first section.

When the sleeve is in an unexpanded state, the outer diameter of the sleeve at its end adjacent to the stent may range from about 0.1 mm to about 1.0 mm, from about 0.25 mm to about 1.0 mm, from about 0.5 mm to about 4 mm, or from about 1.5 mm to about 7 mm. The outer diameter of the sleeve at its end distal to the stent may range from about 0.01 mm to about 1.0 mm, from about 0.5 mm to about 1.5 mm, from about 0.25 mm to about 2.0 mm, or from about 0.25 mm to about 3.0 mm. The outer diameter of the sleeve may be constant across the entire length of the sleeve when the sleeve is in an unexpanded state. The outer diameter of the sleeve may decrease from the end of the sleeve adjacent to the stent to the end of the sleeve distal to the stent when the sleeve is in an unexpanded state. The length of the sleeve may range from about 1 mm to about 7 mm.

Many embodiments of the sleeve may be encompassed by the present invention. For examples, the sleeve may be an O-ring. The sleeve may comprise a plurality of ridges positioned on the outer surface of the sleeve. The sleeve may comprise a ring of grooved indentations on the end of the sleeve adjacent to the stent.

The sleeve may comprise an elastomeric material, such as a high-strength thermoplastic elastomer, including styrenic block copolymers, polyolefin blends, elastomeric alloys, thermoplastic polyurethanes, thermoplastic copolyesters, thermoplastic polyamides, polyester-polyether copolymers and polyamidepolyether copolymers. The thermoplastic polyurethane may have a low durometer grade. The high-strength thermoplastic elastomer may also be nylon. The sleeve may comprise expandable silicone.

The present stent delivery system may further comprise an expandable balloon mounted on the catheter, where the stent is mounted on the balloon. The sleeve may be attached to the catheter and/or balloon. The sleeve may expand and contract radially together with the balloon. The stent that may be used with the present invention include metal stents, biodegradable stents and bioabsorbable stents, as well as coated stents.

### BRIEF DESCRIPTION OF THE DRAWINGS

FIG. 1 shows an example of the balloon catheter system.

FIG. 2a shows the balloon catheter system without the sleeves.

FIG. 2b shows the balloon catheter system with the sleeves.

FIG. 3a shows a close-up view of FIG. 2a.

FIG. 3b shows a close-up view of FIG. 2b.

FIG. 4a shows the balloon catheter system of the present invention with a stent positioned between the two sleeves when the stent is in an unexpanded state.

FIG. 4b shows the balloon catheter system of the present invention with a stent positioned between the two sleeves when the stent is in an expanded state.

FIG. 5 shows an embodiment of the sleeve where the sleeve is an O-ring with a beveled edge.

FIG. 6 shows the embodiment of the sleeve shown in FIG. 5 where the sleeve and a stent is mounted on a balloon catheter.

FIGS. 7a-7d show embodiments of the sleeve where the edge of the sleeve is flanged or flared (FIG. 7a), beveled (FIG. 7b), rounded (FIG. 7e) or straight (FIG. 7d).

FIG. 8a shows an embodiment of the sleeve where the sleeve wall decreases in thickness from the end of the sleeve adjacent to the stent to the end of the sleeve distal to the stent.

FIG. 8b shows a cut-away view of FIG. 8a.

FIG. 9a shows an embodiment of the sleeve where the sleeve wall thickness is constant across the length of the sleeve.

FIG. 9b shows a cut-away view of FIG. 9a.

FIG. 10a shows an embodiment of the sleeve where the sleeve has ridges on the outer surface and along the longitudinal axis of the sleeve.

FIG. 10b shows a cut-away view of FIG. 10a (note, in this embodiment, the sleeve wall decreases in thickness from one end of the sleeve to the other end).

FIG. 11a shows an embodiment of the sleeve where the sleeve has circumferential ridges on the outer surface of the sleeve.

FIG. 11b shows a cut-away view of FIG. 11a.

FIG. 12a shows an embodiment of the sleeve where the sleeve has a ring of grooved indentations on the end of the sleeve adjacent to the stent.

FIG. 12b shows a cut-away view of FIG. 12a.

FIG. 13 shows a close-up view of the sleeve shown in FIG. 12a where the sleeve and a stent is mounted on a catheter.

FIG. 14a shows an embodiment of the sleeve where the sleeve has a plurality of grooved indentations in the inner surface of the sleeve that are configured to fit into corresponding grooves within the body of the catheter or balloon.

FIG. 14b shows a cut-away view of the embodiment shown in FIG. 14a.

FIG. 15a shows a cut-away view of the sleeve embodiment of FIGS. 14a and 14b that is mounted on a balloon catheter.

FIG. 15b shows a close-up view of the sleeve shown in FIG. 15a.

The drawings are not drawn precisely to scale and some dimensions may have been exaggerated for clarity.

### DETAILED DESCRIPTION OF THE INVENTION

The present invention provides one or more sleeves for maintaining the position of a stent on a stent delivery system, such as a balloon catheter. The sleeve wraps around the catheter. One or two sleeves are positioned adjacent to one or both ends of the stent without overlapping with the stent. One end or section of the sleeve may be attached to the catheter or the balloon. In one embodiment, the end of the sleeve that is distal to the stent is attached to the catheter or the balloon. When the sleeve is in an unexpanded state, the outer diameter of the end of the sleeve adjacent to the stent may be equal to or less than the outer diameter of the unexpanded stent. As a result, the profile of the stent delivery system is not increased, by the sleeves of the present invention. The outer diameter of the end of the sleeve adjacent to the stent may also be greater than the outer diameter of the unexpanded stent. If the stent dislodges during delivery to the target site, the stent can come to rest on the wall of the sleeve. When the stent reaches the target site, the balloon is expanded, followed by expansion of the stent and sleeves. Because the sleeves are positioned adjacent to the stent without overlapping with the stent, the stent is able to expand without being restrained by the sleeves. In addition, except for the stent, no other component of the stent delivery system, including the sleeves, comes into contact with the vessel wall when the balloon is expanded. The lack of contact minimizes damages caused by the stent delivery system to the vessel wall, especially the endothelium.

The stent delivery system of the present invention comprises a catheter which has a region for mounting a stent. The stent delivery system further comprises at least one sleeve made from expandable material. The sleeve is open at both ends enclosing a lumen. The sleeve is mounted on the catheter and positioned adjacent to the stent. As used herein, the term “adjacent to” refers to the fact that the sleeve is in close proximity to the stent without overlapping with the stent. In certain embodiments, the sleeve is in direct contact with the stent. The term “outer diameter of the sleeve” (“OD”) refers to the outer diameter of a cross-section of the sleeve, inclusive of the thickness of the sleeve wall. The term “inner diameter of the sleeve” (“ID”) refers to the inner diameter of a cross-section of the sleeve, exclusive of the thickness of the sleeve wall. The term “inner diameter of the sleeve” may alternatively be defined as the diameter of a cross-section of the sleeve lumen.

The stent delivery system of the present invention may have one, or preferably, two sleeves. The stent may be positioned between the two sleeves. The present stent delivery system may comprise three, four, or any number of sleeves that allow for proper positioning of the stent during delivery. The dimension, configuration and shape of the sleeves positioned on the distal or proximal end of the stent delivery system may be the same or may be different. When there are sleeves mounted adjacent to both ends of the stent, the sleeve positioned on the distal end of the stent delivery system is a leading sleeve; the sleeve positioned on the poximal end of the stent delivery system is a trailing sleeve. The term “distal end of the stent delivery system” refers to the end of the stent delivery system that is distal to the Luer fitting or the user of the stent delivery system. The term “proximal end of the stent delivery system” refers to the end of the stent delivery system that is proximal to the Luer fitting or the user of the stent delivery system.

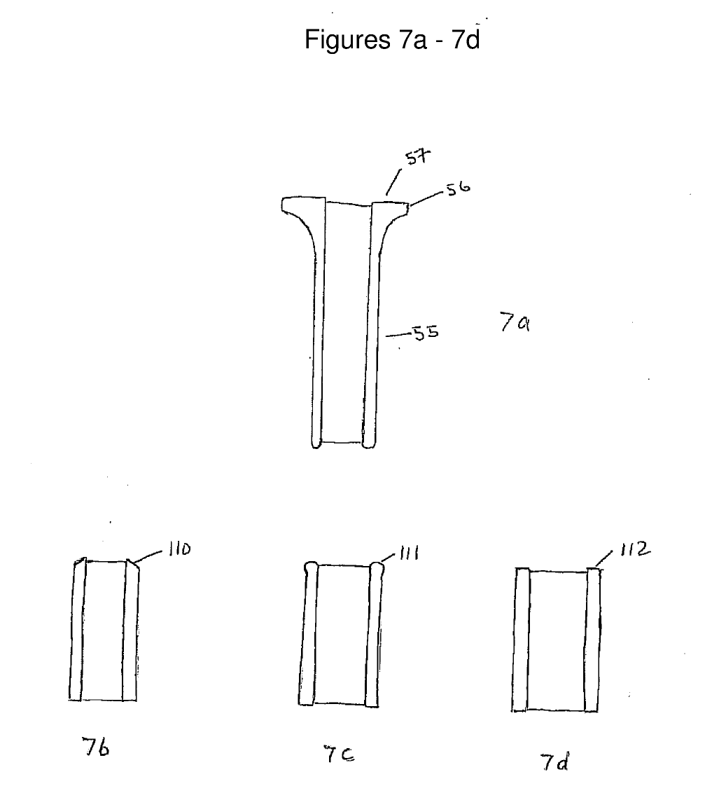

The wall of the sleeve at the end adjacent to the stent may range in thickness from about 0.03 mm to about 0.25 mm, from about 0.03 mm to about 0.2 mm, from about 0.04 mm to about 0.15 mm, or from about 0.04 mm to about 0.1 min. The thickness of the sleeve wall may be constant across the entire length of the sleeve. The thickness of the sleeve wall may vary at various sections of the sleeve. For example, the sleeve wall may decrease in thickness from the end of the sleeve adjacent to the stent to the end of the sleeve distal to the stent. In one embodiment, the wall of the sleeve adjacent to the stent may form a ledge for the stent to rest against if the stent dislodges. The edge of the sleeve adjacent to the stent may be flanged, flared, beveled, rounded or straight (see, FIGS. 7a-7b discussed below). These flanged, flared, beveled, rounded or straight edges may be positioned at either end of the sleeve, i.e., the end adjacent to the stent or the end distal to the stent. The thickness of the sleeve may remain the same or may change during expansion.

When the sleeve is in an unexpanded state, the inner diameter of the sleeve may he constant across the entire length of the sleeve. The inner diameter of the sleeve may vary at different sections of the sleeve. In one embodiment, the inner diameter of the sleeve may decrease from the end adjacent to the stent to the end of the sleeve distal to the stent. This decrease may be linear, step-function, or stepwise progression downwards with multiple shoulders; other decreasing patterns are also possible. In another embodiment, the inner diameter of the sleeve is similar to the outer diameter of the unexpanded (folded) balloon to allow for close contact of the sleeve with the underlying balloon.

When the sleeve is in an unexpanded state, the outer diameter of the sleeve may be constant across the entire length of the sleeve. The outer diameter of the sleeve may vary at different sections of the sleeve. In one embodiment, the outer diameter of the sleeve may decrease from the end adjacent to the stent to the end of the sleeve distal to the stent. This decrease may be linear, step-function, or stepwise progression downwards with multiple shoulders; other embodiments are also possible.

In one embodiment, the inner diameter of the sleeve is constant across a first section of the sleeve beginning from the end of the sleeve adjacent to the stent, then decreasing across a second section of the sleeve adjacent to the first section and then remains constant across a third section adjacent to the second section. The third section being closest to the end of the sleeve distal to the stent; the inner diameter of the third section is less than the inner diameter of the first section.

The length of the sleeve may range from about 1 mm to about 7 mm, from about 1.5 mm to about 6.5 mm, from about 2 mm to about 6 mm, or from about 3 mm to about 5 mm. The length of the sleeve may be chosen by one of ordinary skill in the art such as a physician according to the specific needs of the user based on the patient's vascular profile, physiological and/or biomedical needs, disease state, or the physical characteristics of the guiding (delivery) catheter.

When the sleeve is in an unexpanded state, the outer diameter of the sleeve at its end adjacent to the stent may range from about 0.1 mm to about 1.0 mm, from about 0.25 mm to about 1.0 mm, from about 0.5 mm to about 4 mm, or from about 1.5 mm to about 7 mm. The outer diameter of the sleeve at its end distal to the stent may range from about 0.01 mm to about 1.0 mm, from about 0.5 mm to about 1.5 mm, from about 0.25 mm to about 2.0 mm, or from about 0.25 mm to about 3.0 mm.

The cross-section of the sleeve may be circular, elliptical, oval, oblong, polygonal, rectangular, triangular, or any suitable shape that allows for proper positioning of the stent during delivery. Cross-sections of different areas of the sleeve may be of the same or different shape.

The stent delivery system of the present invention may further comprise an expandable balloon that is mounted on the catheter, where the stent is mounted on the balloon. The sleeve may be attached to the catheter arid/or balloon. When the balloon expands, the sleeve may expand and contract radially together with the balloon.

The sleeve of the present invention may be used with any stent delivery system. In one embodiment, the stent delivery system is a balloon catheter that has a tubular catheter shaft with an inflation lumen and an inflatable balloon attached to the distal end of the catheter shaft. The inflation lumen is in fluid communication with the interior of the balloon The stent delivery system also has a balloon-expandable stent placed around the balloon. The stent is positioned between the proximal and distal ends of the balloon. The stent is expandable from an unexpanded state to an expanded state when inflation pressure is applied to the interior of the balloon. Two sleeves are positioned adjacent to the both ends of the stent.

The following is the description of one type of balloon catheter that may be used with the present sleeve. It should be noted that numerous stent delivery system may be used with the present sleeve.

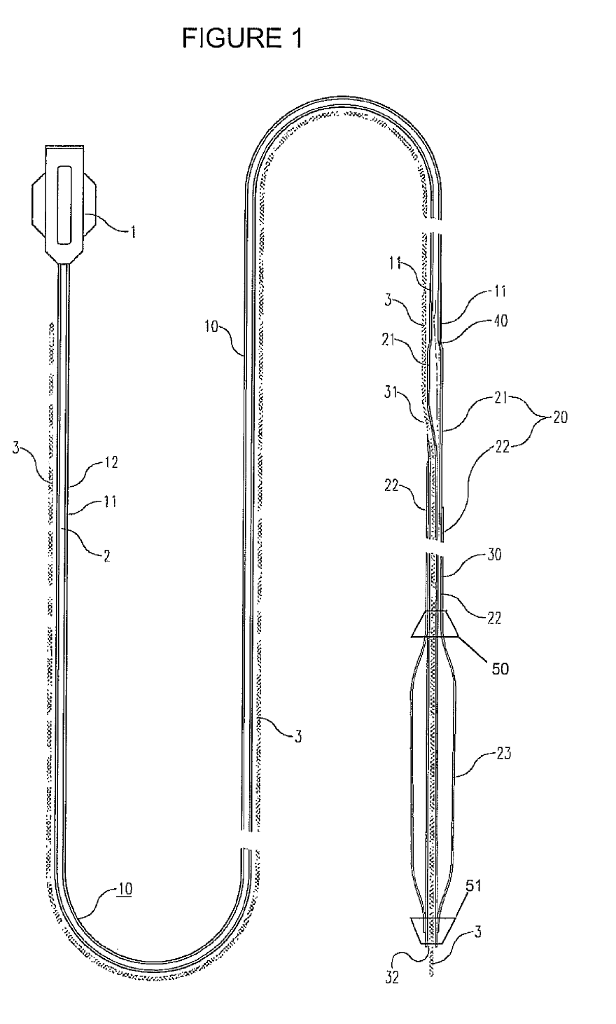

In FIG. 1, the balloon catheter has a distal shaft section 20 and a proximal shaft section 10. The distal shaft section 20 is sufficiently flexible to comply with the natural anatomy of the coronary arteries, whereas the proximal shaft section 10 is more rigid to push and steer the distal section. The proximal shaft section 10 carries at its proximal end a Luer fitting 1 for connecting the catheter to inflation equipment. An inflatable balloon 23 is positioned at the distal end of the catheter. The balloon may be fabricated from a suitable pre-shaped plastic sleeve capable of withstanding a large internal pressure. The inflation equipment enables the delivery of a suitable inflation fluid under pressure and through an inflation lumen 2 extending to the interior of the inflatable balloon 23. A medical practitioner advances the catheter through a guide catheter (not shown) inside the patient's vasculature to the entrance of one of the coronary arteries. On application of inflation pressure, the balloon expands to a predefined diameter to widen the vessel. A stent is positioned on the balloon and is expanded along with the balloon to be deployed in the vessel, thus providing continuous support of the vessel wall after the balloon catheter is retracted. U.S. Pat. No. 7,169,162.

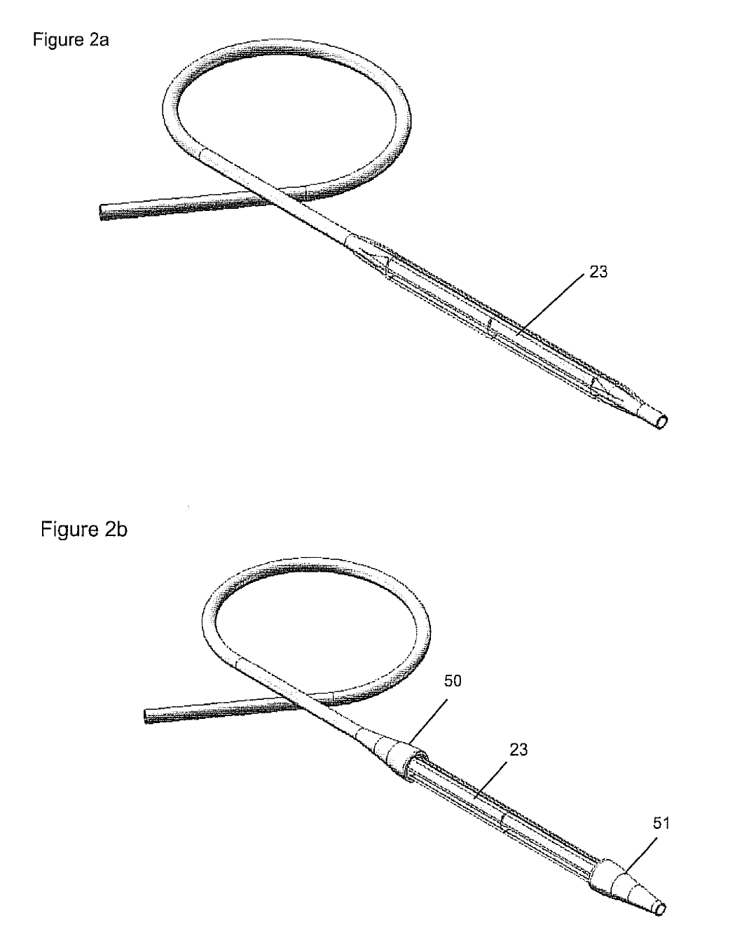

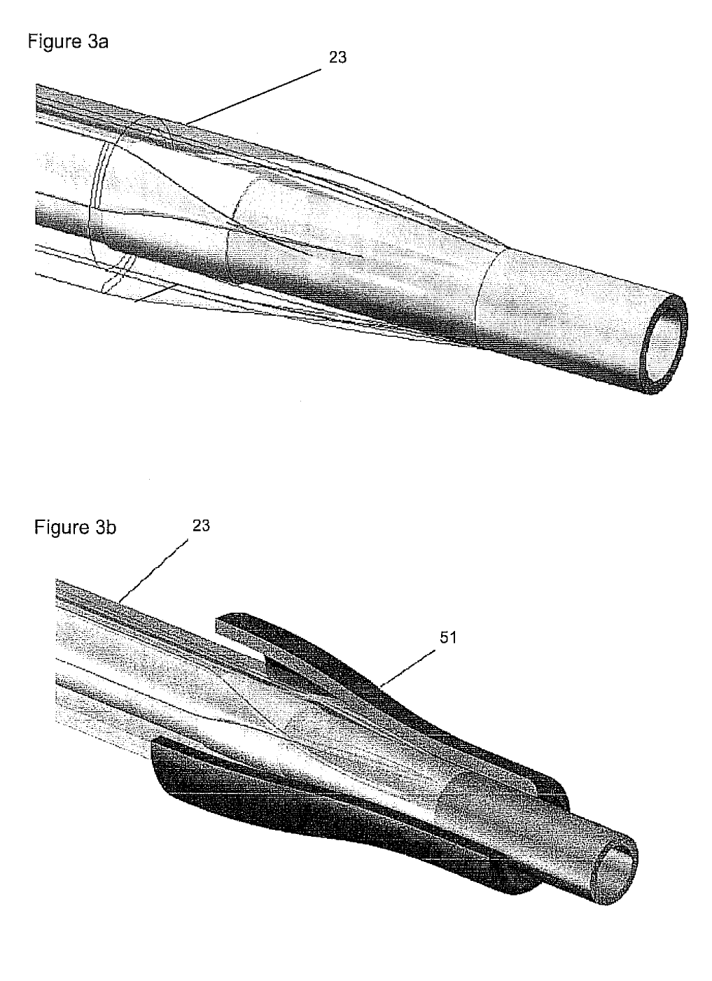

The stent delivery system may have sleeves 50 and 51 that are positioned at the proximal and distal ends of the inflatable balloon 23, respectively. The sleeves are shown in greater detail in FIG. 2b. For comparison purposes. FIG. 2a shows a balloon catheter system without the sleeves and FIG. 3a shows a close-up view of the distal end of the balloon catheter in FIG. 2a, In FIG. 2b, sleeves, 50 and 51, are positioned at the proximal and distal ends of the inflatable balloon 23, respectively. FIG. 3b provides a more detailed illustration of the sleeve in a cut-away diagram. The sleeve wraps around the catheter and may be attached directly to the catheter and/or the balloon. In one embodiment, the end of the sleeve distal to the balloon is attached to the catheter and/or balloon.

The sleeves may be attached to the stent delivery system by various methods such as bonding, gluing, welding or fusing. The sleeve may be attached to the catheter via a suitable adhesive or attachment device. The sleeve may also be attached to the balloon via a suitable adhesive or attachment device. The attachment device may be a mechanical attachment device such as a retaining ring, collar, or any other suitable device that allow for attachment of the sleeve to the catheter and/or balloon. In one embodiment, the sleeve is covalently bound to the balloon or catheter using a cross-linking agent such as glutaraldehyde. Alternatively, the sleeve may be bonded to the catheter and/or the balloon by ultraviolet (“UV”) cross-linking of polymeric material. Methods of attachment may be the same or may vary for the sleeves that are positioned at either end of the stent. More than one method may be combined to attach the sleeve to the catheter and/or balloon.

The sleeve of the present invention is made from expandable material. When the balloon is inflated, the sleeve also expands. The expandable material is chosen such that after the balloon is deflated, the retention sleeve returns to its unexpanded state without being plastically deformed, broken, torn, inverted or rolled back onto the sleeve itself. In one embodiment, the expandable material is constructed of expandable silicone. In another embodiment, the expandable material is an elastomer. Preferably, the elastomer is a high-strength thermoplastic elastomer. This high-strength thermoplastic elastomer can be a styrenic block copolymer, a polyolefin blend, an elastomeric alloy, a thermoplastic polyurethane, a thermoplastic copolyester, or a thermoplastic polyamide. In a further embodiment, the high-strength thermoplastic elastomer is a thermoplastic polyurethane. Preferably, this thermoplastic polyurethane has a low durometer grade. For example, the thermoplastic polyurethane has a durometer grade of between 40-50 A. In still another embodiment, the high-strength thermoplastic elastomer is a polyester-polyether copolymer or a polyamide-polyether copolymer. In yet another embodiment, the high-strength thermoplastic elastomer is nylon. Various grades of nylon may be used for fabricating the sleeve. The sleeve of the present invention may he made of one or more thermoplastic elastomers, such as block copolymers; copolymers and terpolymers of ethylene; homopolymers, copolymers and terpolymers of propylene ethylene a-olefins; polyesters; polyamides; polyurethanes, such as TECOTHANE™ (biocompatible medical grade aromic polyurethane available from Thermedics, Inc.); polycarbonates, vinyl copolymers; ionomer materials and so forth. More specifically, materials such as SELAR™, polyether-polyester block copolymers (i.e. HYTRBL™ from DuPont or ARNITEL™ from DSM, Netherlands), PEBAX™ (polyether block amide copolymers), SURLYN™, polyethylene terephthalate, polytetrafluoroethylene, polyvinyl chloride, polyetherurethanes, polyesterurethanes, polyurethane ureas, polyurethane siloxane block copolymers, silicone polycarbonate copolymers, ethylene vinyl acetate copolymers, acrylonitrile-butadiene-styrene copolymers polyphenylene sulfides, copolyesters or other similar extrudable thermoplastic, polymeric materials and/or composites thereof may be utilized in the present invention. U.S. Pat. Nos. 6,547,813, 6,565,595 and 6,805,702.

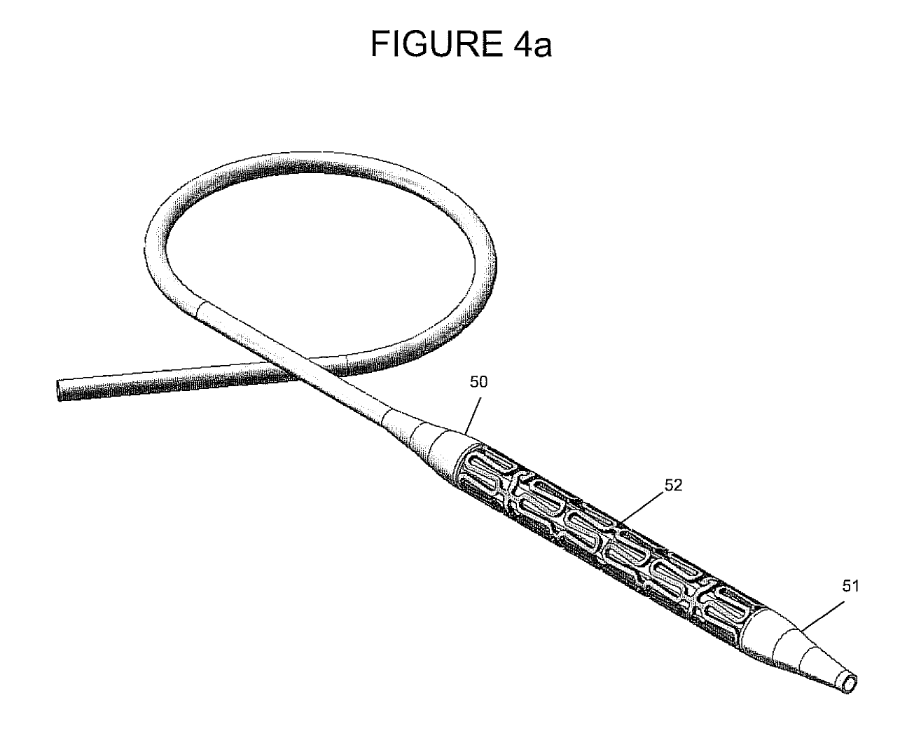

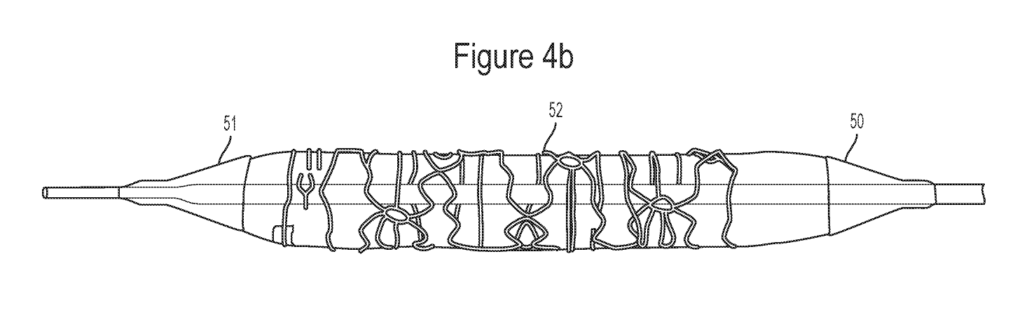

FIG. 4a shows the balloon catheter system of the present invention when the stent is in an unexpanded state. The stent 52 is mounted on the catheter and positioned between two sleeves 50, 51. Sleeves 50 and 51 are adjacent to stent 52 without overlapping with the stent. The sleeve wraps around the catheter and may be attached directly to the catheter or balloon. In this embodiment, the outer diameter of the end of the unexpanded sleeve adjacent to the stent is equal to or less than the outer diameter of the unexpanded stent. As a result, the profile of the stent delivery system is not increased by the sleeves of the present invention. During deployment, the stent delivery system is inserted into a vessel. As stent 52 reaches the target site, balloon 23 is expanded by the inflation fluid supplied to the balloon lumen, followed by expansion of stent 52 and sleeves 50, 51 (FIG. 4b). Because sleeves 50 and 51 are positioned adjacent to stent 52 without overlapping with the stent, stent 52 is able to expand during deployment without being restrained by the sleeve. The outer diameter of the expanded sleeve at the end adjacent to the stent is less than the outer diameter of the expanded stent. Therefore, when the balloon is in an expanded state, except for stent 52, no other component of the stent delivery system, including the sleeves, comes into contact with the vessel wall. In this embodiment, the sleeves lay on the shoulder of the balloon when the balloon is expanded. This lack of contact with the vessel wall minimizes damage to the vessel, especially the vascular endothelium. After steal 52 is deployed, the balloon is deflated. The sleeves 50, 51 contract radially together with the balloon and return to their unexpanded state. The catheter with sleeves 50, 51 and balloon 23 is then removed from the vessel, leaving stent 52 implanted in the body.

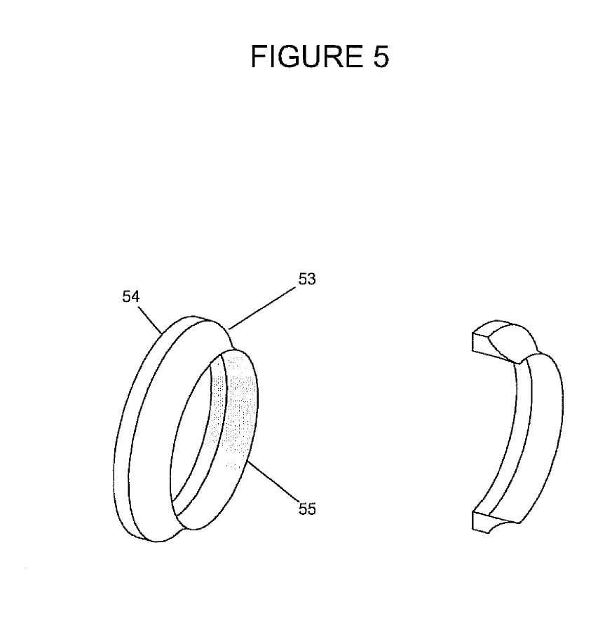

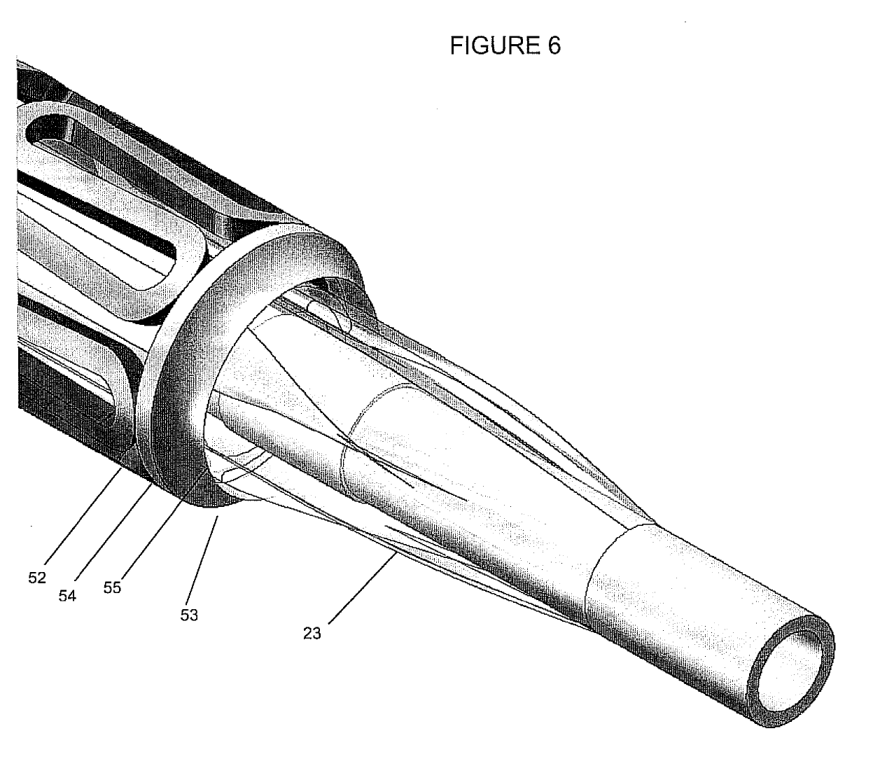

Many different embodiments of the sleeve are encompassed by the invention. In one embodiment, the sleeve may be an O-ring (FIG. 5). The lip 55 of of the O-ring sleeve 53 is positioned distal to stent 52 in the assembled stent delivery system (FIG. 6). The O-ring sleeve 53 is positioned over the inflatable balloon 23. The stent delivery system may comprise one or more O-rings positioned on either side of the stent.

The edge of the sleeve adjacent to the stent may be flanged (FIG. 7a), beveled (FIG. 7b), rounded (FIG. 7c) or straight (FIG. 7d). These flanged 56, beveled 110, rounded 111 or straight 112 edges may be positioned at either end of the sleeve, i.e., the end adjacent to the stent or the end distal to the stent. The edge of the sleeve may adopt any suitable configuration that allows for proper positioning of the stent during delivery.

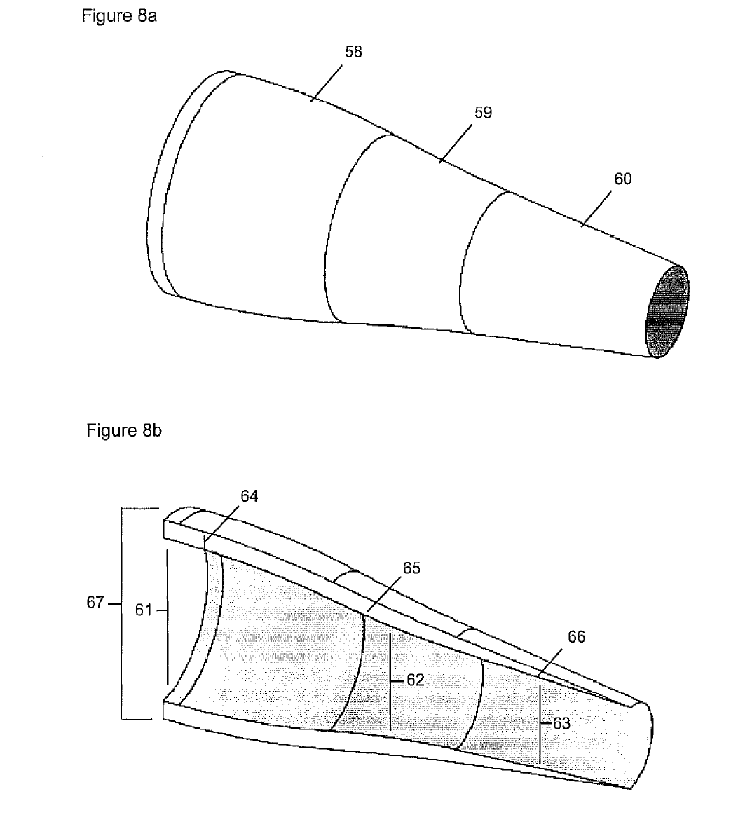

In another embodiment, the outer diameter and inner diameter of the sleeve decreases from its end adjacent to the stent to the end of the sleeve distal to the stent (FIGS. 8a and 8b). In FIG. 8a, the sleeve comprises three sections 58, 59 and 60, with section 58 being adjacent to the stent. The inner diameter of the sleeve decreases across the length of the sleeve (see, 61, 62 and 63, where the inner diameter is such that 61>62>63). The sleeve wall decreases in thickness from the end adjacent to the stent to the end distal to the stent (see, 64, 65 and 66, where the thickness of the wall is such that 64>65>66).

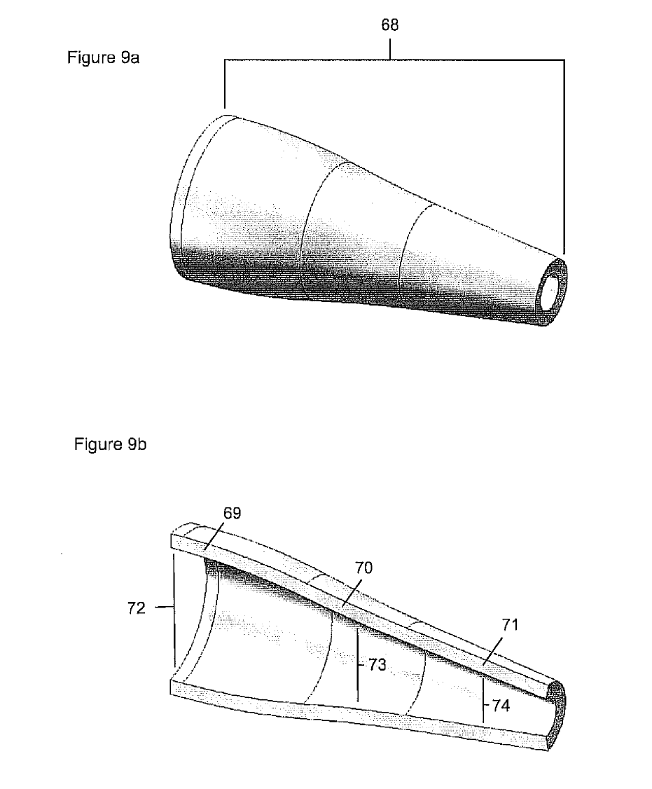

In a further embodiment shown in FIG. 9, the inner diameter of the sleeve decreases across the length of the sleeve (see, 72, 73 and 74, where the inner diameter is such that, 72>73>74). The thickness of the sleeve wall is constant across the entire length of the sleeve (see, 69, 70 and 71, where the thickness of the wall is such that, 69=70=71).

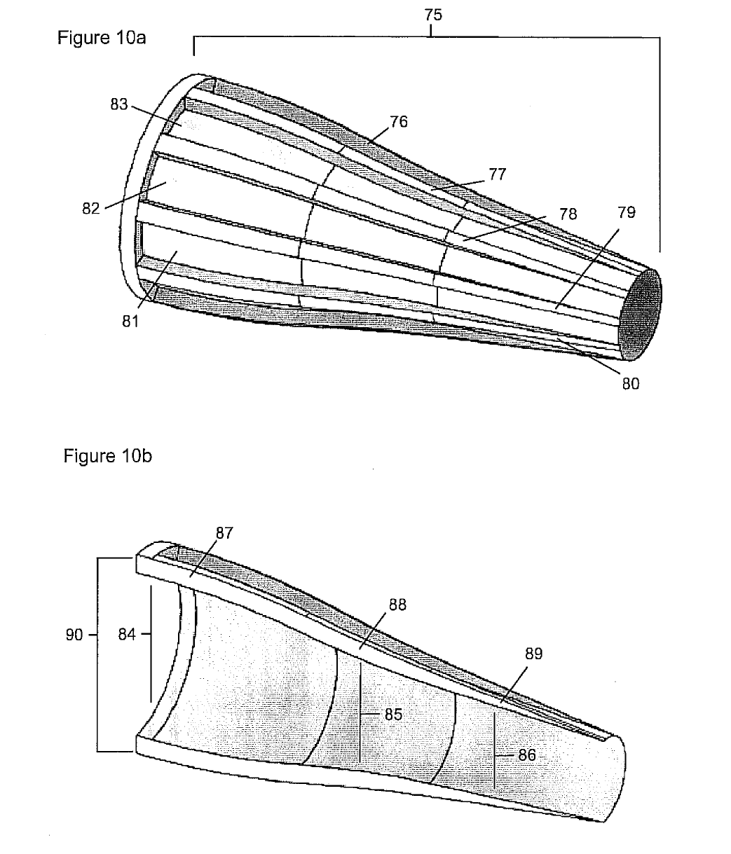

In yet another embodiment, the sleeve contains a plurality of ridges on its outer surface (FIG. 10a). The sleeve 75 has a plurality of ridges, 76-80, positioned along the longitudinal axis of the sleeve. The ridges enclose spaces 81-83. The dimension of the ridges may vary or may be the same. The sleeve wall decreases in thickness across the length of the sleeve (see, 87, 88 and 89 in FIG. 10b, where the thickness of the wall is such that, 87>88>89). The inner diameter of the sleeve decreases across the length of the sleeve (see 84, 85 and 86, where the inner diameter is such that, 84>85>86). The end of the sleeve adjacent to the stent has the largest inner diameter 84 and outer diameter 90.

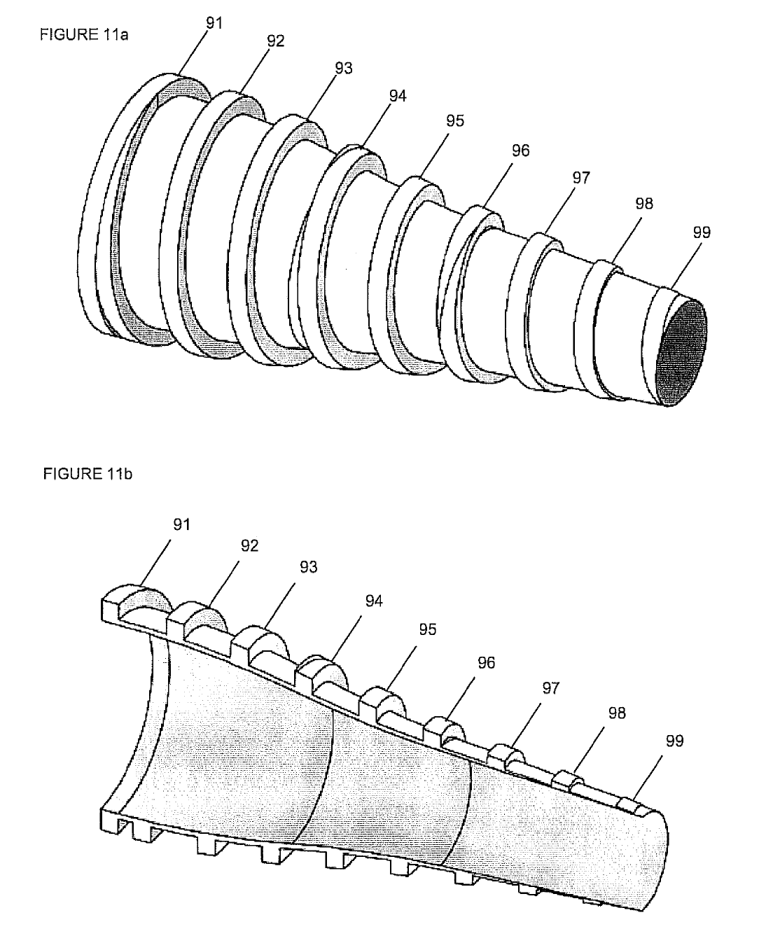

In still another embodiment, the sleeve has a plurality of circumferential, ridges, 91-99, positioned on the outer surface and along, the axial axis of the sleeve (FIGS. 11a and 11b). These circumferential ridges may be discrete or may form a helical or spiral path around the circumference of the sleeve.

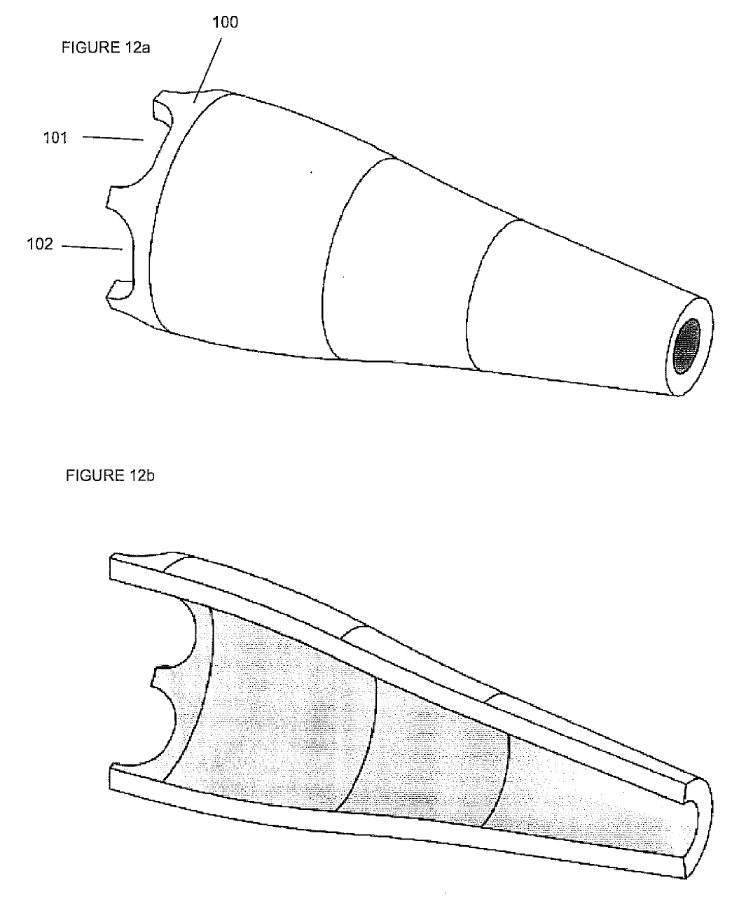

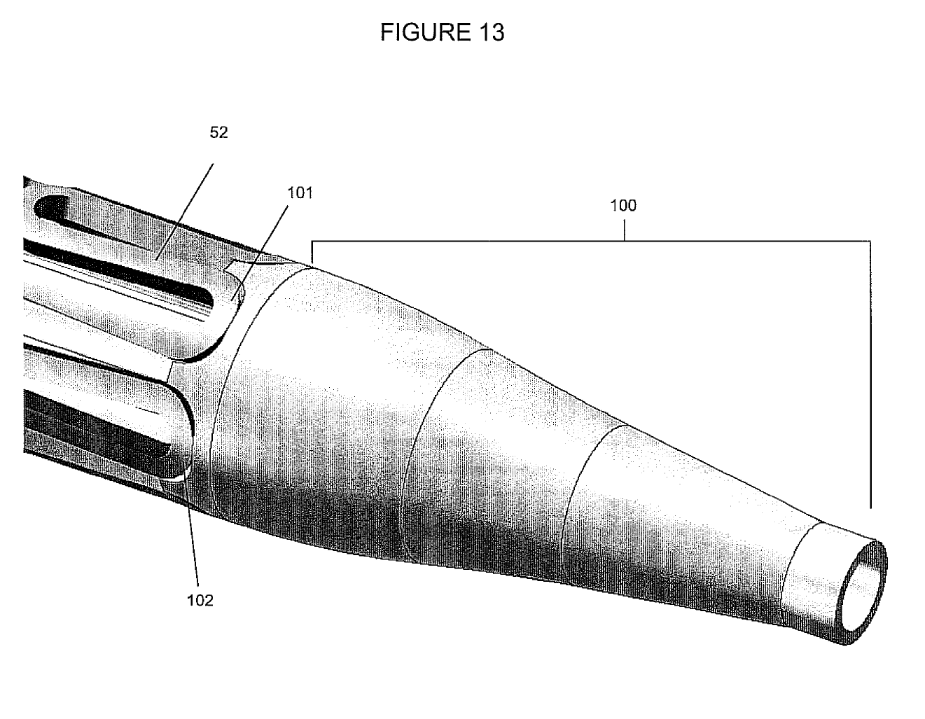

The sleeve may comprise a ring of grooved indentations, 101, 102, on the end of the sleeve adjacent to the stent. These grooved indentations allow for positioning of the ends of the stent in the sleeve (FIGS. 12a and 12b). These grooved indentations can be constructed to take any shape, e.g., S-shaped, C-shaped, H-shaped, sinusoid, in order to accommodate a wide variety of stent shapes. The contour of the end of the stent 52 may fit within the grooved indentations, 101 and 102, of the sleeve 100 to ensure proper positioning of the stent (FIG. 13).

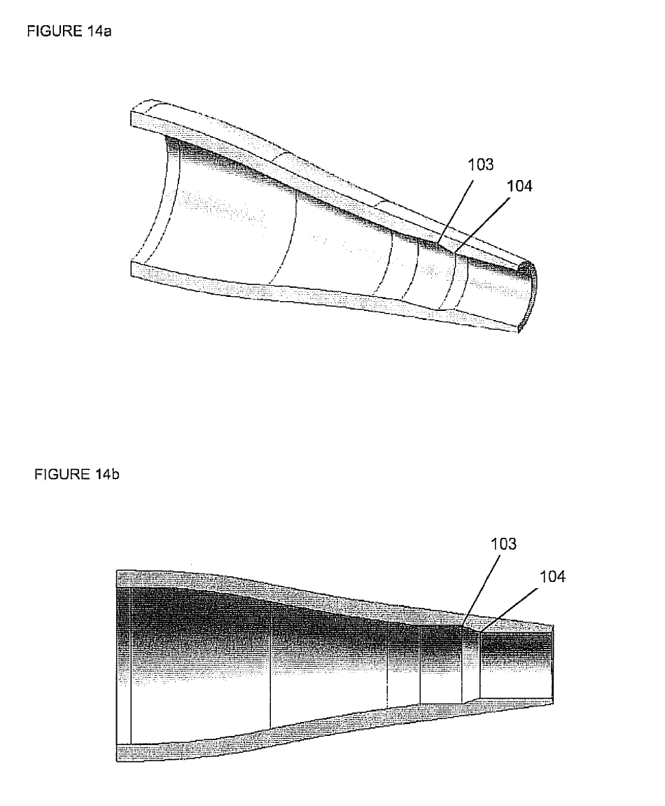

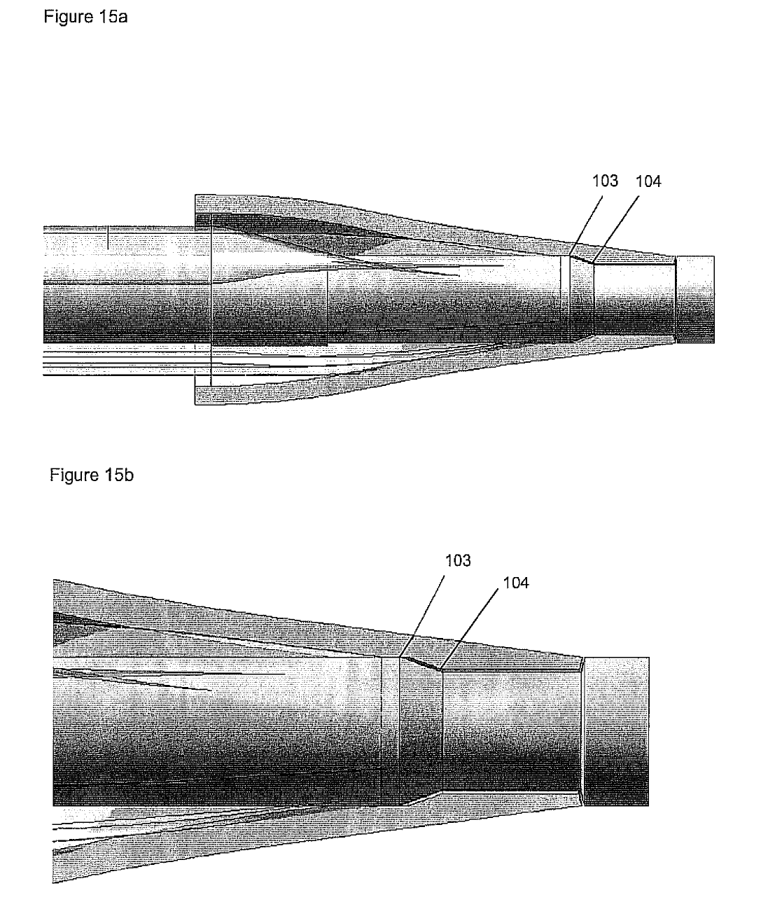

In FIGS. 14 and 15, the sleeve may have a plurality of grooved indentations 103 and 104 in the inner surface of the sleeve that are configured to fit into corresponding grooves in the body of the catheter or balloon that is in contact with the inner surface of the sleeve. The fitting of the grooves of the catheter with the grooves of the sleeve is best appreciated in FIGS. 15a and 15b.

The sleeve of the present invention may be coated with a lubricant on its inner surface, or on both its inner and outer surfaces, before the sleeve is assembled with the catheter and/or after the sleeve is assembled with the catheter. The lubricant may be added to the sleeve material during extrusion. The lubricant may also be compounded with the sleeve material prior to extrusion. All of these lubrication mechanisms can be combined for maximum effectiveness. The lubricant coating the sleeve may be hydrophobic and/or hydrophilic and may be selected from, but are not limited to, one or more of the following substances: silicones; PVP (polyvinyl pyrrolidone); PPO (polypropylene oxide); PEO; BioSlide™ coating (a hydrophilic lubricious coating produced by SciMed, which comprises polyethylene oxide and neopentyl glycol diacrylate polymerized in a solution of water and isopropyl alcohol in the presence of a photoinitiator such as azobisisobutronitrile); oils, such as mineral oil, olive oil, vegetable oil, or other natural oils and wax. A number of lubricants may be added into elastomer or thermoplastic compositions during melt processes or compounding, such as fluoropolymer powders, graphite, fatty acid esters and amides, hydrocarbon waxes and silicone masterbatch additive. U.S. Pat. Nos. 6,221,097, 6,331,186 and 6,443,980.

The sleeve of the present invention may be used with any stent delivery system, such as the balloon catheter stent delivery systems described in U.S. Pat. Nos. 6,168,617, 6,222,097, 6,331,186 and 6,478,814. The stent may be self-expanding, such as a nitinol shape memory stent. The stent may also be expandable by means of an expandable portion of the catheter, such as a balloon. The stent that may be used in the present invention, include metal stents, biodegradable stents and bioabsorbable stents, as well as coated stents.

The sleeve of the present invention may be used with any suitable catheter, the diameter of which may range from about 0.8 mm to about 5.5 mm, from about 1.0 mm to about 4.5 mm, from about 1.2 mm to about 2.2 mm, or from about 1.8 to about 3 mm. In one embodiment, the catheter is about 6 French (2 mm) in diameter. In another embodiment, the catheter is about 5 French (1.7 mm) diameter.

The present invention can be used for any vessel such as any artery or vein. Included within the scope of this invention is any artery including coronary, infrainguinal, aortoiliac, subclavian, mesenteric and renal arteries. Other types of vessel obstructions, such as those resulting from a dissectinv, aneurysm are also encompassed by the invention. The present invention can further be used for any conduit or cavity in mammals. The subjects that can be treated using the stent and devices of this invention are mammals, including a human, horse, dog, cat, pig, rodent, monkey and the like.

The scope of the present invention is not limited by what has been specifically shown and described hereinabove. Those skilled in the art will recognize that there are suitable alternatives to the depicted examples of materials, configurations, constructions and dimensions. The materials and dimensions used in the foregoing embodiment may be replaced by other existing or newly developed materials; other dimensions may be used that offer the best practical performance. Numerous references, including patents and various publications, are cited in the description of this invention. The citation and discussion of such references is provided merely to clarify the description of the present invention and is not an admission that any reference is prior art to the invention described herein. All references cited and discussed in this specification are incorporated herein by reference in their entirety. While certain embodiments of the present invention have been shown and described, it will be obvious to those skilled in the art that changes and modifications may be made without departing from the spirit and scope of the invention. The matter set froth in the foregoing description and accompanying drawings is offered by way of illustration only and not as a limitation. Specific terminology is employed only for the sake of clarity. However, the invention is not intended to be limited to the specific terminology selected. It is to be understood that each specific element includes all technical equivalents that operate in a similar or like manner to accomplish a similar purpose.

1. A stent delivery system comprising a catheter which has a region for mounting a stent and at least one sleeve comprising expandable material, wherein the sleeve is mounted on the catheter and where the sleeve is positioned adjacent to the stent.

2. The stent delivery system of claim 1, wherein the sleeve is in direct contact with the stent.

3. The stent delivery system of claim 1, wherein the outer diameter of the end of the unexpanded sleeve adjacent to the stent is equal to the outer diameter of the unexpanded stent mounted on the catheter.

4. The stent delivery system of claim 1, wherein the outer diameter of the end of the unexpanded sleeve adjacent to the stent is less than the outer diameter of the unexpanded stent mounted on the catheter.

5. The stent delivery system of claim 1, wherein the sleeve wall decreases in thickness from the end of the sleeve adjacent to the stent to the end of the sleeve distal to the stent.

6. The stent delivery system of claim 1, wherein the thickness of the sleeve wall is constant across the length of the sleeve.

7. The stent delivery system of claim 1, wherein the inner diameter of the sleeve is constant across the entire length of the sleeve when the sleeve is in an unexpanded state.

8. The stent delivery system of claim 1, wherein the inner diameter of the sleeve decreases from the end of the sleeve adjacent to the stent to the end of the sleeve distal to the stent when the sleeve is in an unexpanded state.

9. The stent delivery system of claim 1, wherein the inner diameter of the sleeve is constant across a first section of the sleeve beginning from the end of the sleeve adjacent to the stent, then decreases across a second section of the sleeve adjacent to the first section and is constant across a third section adjacent to the second section, the third section being closest to the end of the sleeve distal to the stent and wherein the inner diameter of the third section is less than the inner diameter of the first section.

10. The stent delivery system of claim 1, wherein the outer diameter of the sleeve is constant across the entire length of the sleeve when the sleeve is in an unexpanded state.

11. The stent delivery system of claim 1, wherein the outer diameter of the sleeve decreases from the end of the sleeve adjacent to the stent to the end of the sleeve distal to the stent when the sleeve is in an unexpanded state.

12. The stent delivery system of claim 1, wherein the sleeve comprises a plurality of ridges on the outer surface of the sleeve.

13. The stent delivery system of claim 1, wherein the sleeve comprises a ring of grooved indentations on the end of the sleeve adjacent to the stent.

14. The stent delivery system of claim 1, wherein the sleeve comprises an elastomer.

15. The stent delivery system of claim 1, wherein the sleeve comprises expandable silicone.

16. The stent delivery system of claim 1, wherein the length of the sleeve ranges from about 1 mm to about 7 mm.

17. The stent delivery system of claim 1, wherein the sleeve is attached to the catheter.

18. The stent delivery system of claim 1, further comprising an expandable balloon, wherein the balloon is mounted on the catheter and where the stent is mounted on the balloon.

19. The stent delivery system of claim 18, wherein the sleeve is attached to the balloon.

20. The stent delivery system of claim 1, wherein there are two sleeves, the stent being positioned between the two sleeves.
